(19) United States
(12) Patent Application Publication
(10) Pub. No.: US 2024/0374555 A1
(43) Pub. Date: Nov. 14, 2024

(54) Mast Cell Stabilizer with Sodium Cromoglycate-based Inhibitor for Preventing Lipedema Progression

(71) Applicants: Gabriele Bonetti, San Felice del Benaco (Brescia) (IT); Matteo Bertelli, San Felice del Benaco (Brescia) (IT); Kevin Donato, San Felice del Benaco (Brescia) (IT); Maria Chiara Medori, San Felice del Benaco (Brescia) (IT); Kristjana Dhuli, Tirana (AL); Cecilia Micheletti, San Felice del Benaco (Brescia) (IT); Jurgen Kaftalli, Tirana (AL); Sandro Michelini, Rome (IT); Karen Louise Herbst, Tucson, AZ (US); Simone Cristoni, San Felice del Benaco (Brescia) (IT)

(72) Inventors: Gabriele Bonetti, San Felice del Benaco (Brescia) (IT); Matteo Bertelli, San Felice del Benaco (Brescia) (IT); Kevin Donato, San Felice del Benaco (Brescia) (IT); Maria Chiara Medori, San Felice del Benaco (Brescia) (IT); Kristjana Dhuli, Tirana (AL); Cecilia Micheletti, San Felice del Benaco (Brescia) (IT); Jurgen Kaftalli, Tirana (AL); Sandro Michelini, Rome (IT); Karen Louise Herbst, Tucson, AZ (US); Simone Cristoni, San Felice del Benaco (Brescia) (IT)

(73) Assignee: MAGI'S LAB SRL, Rovereto (Trento) (IT)

(21) Appl. No.: 18/630,412

(22) Filed: Apr. 9, 2024

Related U.S. Application Data
(60) Provisional application No. 63/495,155, filed on Apr. 10, 2023.

(51) Int. Cl. A61K 31/353

(52) U.S. Cl. CPC A61K 31/353

(57) ABSTRACT

Disclosed is a pharmaceutical inhibitor for the treatment of lipedema in a subject. The inhibitor comprises a mast cell stabilizer comprising sodium cromoglycate, but can be selected from a group of compounds that includes nedocromil, ketotifen, disodium chromoglycate, olopatadine, and amlexanox. The inhibitor can be administered as a composition or topical cream. The dosage of sodium cromoglycate can be about 500 mg for the composition or a 4% concentration for the topical cream. The treatment regimen can involve oral administration of the sodium cromoglycate composition or epicutaneous administration of the sodium cromoglycate cream. The treatment reduces histamine levels and mast cell degranulation, thereby inhibiting the growth and proliferation of adipose tissue cells, which prevents an increase in the size or mass of the tissue. This invention provides a new approach for treating lipedema, which can improve the quality of life for individuals affected by this condition.

### TECHNICAL FIELD OF THE INVENTION

The technical field of the invention is pharmaceuticals and medicine, specifically the treatment of lipedema using mast cell stabilizers. More specifically, it relates to inhibiting the growth and proliferation of adipose tissue cells in individuals affected by lipedema.

### BACKGROUND ART

Lipedema is a chronic, often debilitating disease that is characterized by excess subcutaneous adipose tissue (SAT) in the arms and legs, as well as bruising and pain. It primarily affects women but has been reported in men as well. Lipedema often appears during times of hormone change, such as puberty or menopause, and can cause significant mobility issues and discomfort if left untreated.

Due to the excess SAT, lipedema is often confused with obesity, and frequently coexists with obesity. However, there are several key differences between the two conditions. One of the most notable differences is the distribution of body fat SAT. In lipedema, the excess SAT is primarily located in the lower limbs and upper extremities, sparing the upper trunk (chest, upper back). In contrast, obesity tends to affect the entire body, especially the trunk. Another key difference is that lipedema tissue does not typically respond well to diet and exercise interventions or bariatric surgery. While weight loss can be achieved through these methods in obesity, people with lipedema may find that these interventions have little effect on the fat accumulation in their legs and arms. Finally, women with lipedema can have a normal Body Mass Index (BMI) despite having an excess of SAT in their lower limbs and upper extremities. This is because BMI is calculated based on an individual's weight and height and does not take into account fat distribution on the body.

In lipedema, there is a disorder of lipid metabolism resulting in the symmetrical deposition of SAT associated with hyperplasia and/or hypertrophy of fat cells. While the etiopathogenesis of lipedema is unclear, it may be associated with impairment of a hormonal axis, since lipedema mainly affects females, and its onset is primarily around the time of puberty.

There is evidence that lipedema may have an underlying genetic component. Several studies found that people with lipedema are more likely to have family members who also have the condition, suggesting a heritable component to lipedema. There is also evidence to suggest that certain molecular mechanisms may play a role in the development and progression of lipedema. For example, some studies found that people with lipedema have abnormal levels of certain hormones, such as leptin and insulin, which may contribute to the accumulation of SAT in the legs and arms. Other research indicates that people with lipedema may have abnormalities in their lymphatic system, the network of vessels that helps drain fluid from the body interstitial space. These abnormalities may prevent proper drainage of fluids, leading to the accumulation of SAT and fluid in the legs and arms.

Recently, researchers discovered the first gene associated with lipedema: ARK1C1, a gene encoding an aldo ketoreductase. The identification of AKR1C1 as a mutated gene in lipedema was the result of a multi-year research effort involving collaboration between multiple scientific teams. The researchers used advanced genetic sequencing techniques to identify mutations in the AKR1C1 gene in a large group of people with lipedema. They found these mutations were present in a significantly higher proportion of people with lipedema compared to people without the condition. The AKR1C1 gene plays a critical role in the metabolism of fatty acids and steroids, and mutations in this gene have been linked to several other diseases, including prostate cancer and obesity. The discovery that AKR1C1 is mutated in people with lipedema confirms the importance of genetics in this disease and suggests that abnormalities in the metabolism of fatty acids and steroids may play a role in the development and progression of this condition. The enzyme aldo-keto reductase is also responsible for the metabolism of progesterone. Estrogen and progesterone are known to affect the deposition of SAT. When girls undergo puberty, they deposit more SAT on their hips and thighs. Estrogen regulates body energy homeostasis and plays a role in the metabolism of fat including subcutaneous fat. Progesterone also reverses the weight reduction actions of estradiol.

Estrogen, a hormone produced primarily in the ovaries and in smaller amounts by the adrenal gland and adipose tissue, is a vital hormone for proper functioning of female reproductive system and plays a key role in the development of secondary sexual characteristics in women. Estrogen levels fluctuate throughout a woman's menstrual cycle and can also be affected by factors such as pregnancy, menopause, and certain medications. Estrogen has a positive direct effect on adipose tissue through estrogen receptors (ERs). Rarely, men are affected with lipedema. Men who develop lipedema tend to be hypogonadal or have liver disease resulting in relatively elevated estrogen levels. Estrogen may, therefore, play a role in the development of adipose and other tissues that have sex hormone receptors. Additional mechanisms thought to play a role in the pathogenesis of lipedema include increased vascular/or permeability and damage (microangiopathy).

Estrogen, a key regulator of adipocyte lipid and glucose metabolism, and female-associated body fat distribution are postulated to play a contributory role in the pathophysiology of lipedema. Dysregulation of adipose tissue accumulation via estrogen signaling likely occurs by two mechanisms: (1) altered adipocyte estrogen receptor distribution (ERα/ERβ ratio) and subsequent metabolic signaling and/or (2) increased release of adipocyte-produced steroidogenic enzymes leading to increased paracrine estrogen release.

There is evidence to suggest that there may be a correlation between estrogens and the organic nitrogenous compound, histamine, two substances that play important roles in the body. Histamine is produced by cells of the body and impacts several important physiological processes, including immune function and the regulation of blood pressure. Several studies suggest that estrogens affect histamine levels in the body. For example, research discovered that estrogen levels correlate with histamine levels in the brain. Other studies suggest that estrogens may influence the release of histamine from immune cells, and that they may affect the activity of enzymes involved in the metabolism of histamine. There is also anecdotal evidence suggesting that histamine may be involved in the development and progression of lipedema. Some studies found that people with lipedema have abnormal levels of histamine, and that the symptoms of lipedema may be correlated with histamine levels.

Estrogen receptors are protein structures found on the surface of cells that play a critical role in the regulation of estrogen-dependent processes in the body. Estrogen receptors are present on a variety of cell types, including mast cells, a type of immune cell involved in allergic reactions and immune defense. The presence of estrogen receptors in mast cells suggests that these cells may be influenced by estrogen, and that estrogen may play a role in the function of mast cells. For example, some research suggests that estrogen may affect the release of histamine from mast cells, and that it may influence the activation and migration of these cells.

The cycle of increased estrogen production and mast cell activation in adipose tissue can be self-perpetuating, leading to a vicious cycle of inflammation and tissue proliferation. For example, higher levels of estrogen in adipose tissue stimulates the recall of mast cells. When these mast cells are activated, they degranulate and release a variety of inflammatory cytokines and growth factors. These substances stimulate the growth and proliferation of adipocytes, as well as further increase estrogen production. As an hypothesis, his feedback loop of excess estrogen, increased mast cell activation and inflammation, proliferation of adipose tissue and increased estrogen production could characterize people with lipedema, and could lead to the worsening of lipedema symptoms and the development of other related health problems. New studies should address this hypothesis and check if the estrogens levels are different between pathologic and healthy subcutaneous adipose tissue in people with lipedema, and if adipocytes and/or mast cells have more receptors for estrogens.

In this scenario, we could consider lipedema as a disease in which the hormonal balance in the adipose tissue is disrupted, leading to an excess of estrogen production. This excess estrogen leads to the recall and degranulation of mast cells, which become a key element in the development and progression of the disease. The inflammation and tissue proliferation that result from the activation of mast cells can lead to the characteristic symptoms of lipedema, such as increased volume of adipocytes and enlargement of the limbs.

Mast cells are known to play a role in a variety of immune and inflammatory processes in the body, and their activation in adipose tissue may contribute to the development of other associated health problems, such as lymphatic dysfunction and insulin resistance. Therefore, understanding the role of mast cells in the pathogenesis of lipedema may be important for developing effective treatments for this condition.

Many of the symptoms of lipedema can be traced back to the activation of mast cells in adipose tissue. The proliferation of adipose tissue, which leads to limbs' enlargement, could be caused by the inflammation and tissue damage that results from mast cell activation. Moreover, the pain, inflammation, and other discomfort commonly experienced by people with lipedema may also be related to the presence of activated mast cells in the affected tissues.

The symptoms of lipedema vary from person to person but may include swelling and bruising of the legs and arms, discomfort and pain, and difficulty with mobility. Pain is the leading symptom in lipedema, and it is usually associated with depression and impaired quality of life. In severe cases, lipedema can lead to difficulty with everyday activities such as walking, standing, or even sitting. There is currently no cure for lipedema, but the condition can be managed through a combination of diet, exercise, and specialized medical treatment.

Diet and exercise can help to reduce overall body fat and improve mobility but are typically not effective at reducing the lipedema specific fat accumulation in the legs and arms. Medical treatments for lipedema may include manual lymphatic drainage, compression therapy, and liposuction. Currently, there are no drugs specifically approved for the treatment of lipedema.

### OBJECT OF THE INVENTION

The object of the invention is to provide a new and effective treatment for lipedema, a chronic condition characterized by the abnormal accumulation of fat tissue in specific areas of the body. The invention uses sodium cromoglycate, a mast cell stabilizer, but may include a selection from nedocromil, ketotifen, disodium chromoglycate, olopatadine, and amlexanox, to inhibit the growth and proliferation of adipose tissue cells in individuals affected by lipedema.

### SUMMARY OF THE INVENTION

The following summary is an explanation of some of the general inventive steps for the treatment composition in the description. This summary is not an extensive overview of the invention and does not intend to limit its scope beyond this summary.

The present invention relates to a pharmaceutical inhibitor for treating lipedema in a subject, using mast cell stabilizers, specifically sodium cromoglycate, for treating lipedema in a subject, although other options such as nedocromil, ketotifen, disodium chromoglycate, olopatadine, and amlexanox may also be considered.

The invention provides a method of manufacture comprising the steps of identifying an inhibitor to an active ingredient, adding an effective amount of a mast cell stabilizer, and mixing the preparation.

The invention also provides a method of using mast cell stabilizers as inhibitors for treating the proliferation of lipedema, by reducing histamine levels and mast cell degranulation, and inhibiting the growth and proliferation of cells within the adipose tissue, thereby preventing an increase in its size. By reducing histamine levels and mast cell degranulation, the invention prevents an increase in the size or mass of the tissue, thus improving the quality of life for individuals affected by this condition.

The inhibitor can be administered in different forms, including compositions and topical creams, and can be orally administered or administered via the epicutaneous route. The invention provides an effective and novel treatment for lipedema, improving the quality of life for individuals affected by this condition.

### CITED REFERENCES

1. Aksoy, H., Karadag, A. S. & Wollina, U. Cause and management of lipedema-associated pain. Dermatologic Therapy vol. 34 Preprint at https://doi.org/10.1111/dth.14364 (2021).
2. Poojari, A., Dev, K. & Rabiee, A. Lipedema: Insights into Morphology, Pathophysiology, and Challenges. Biomedicines vol. 10 Preprint at https://doi.org/10.3390/biomedicines10123081 (2022).
3. S Paolacci, V. P. F. A. P. C. E. F. M. P. F. B. S. M. K. L. H. V. U. M. B. G. P. Genetics of lipedema: new perspectives on genetic research and molecular diagnoses. (2019).
4. Michelini, S. et al. Aldo-keto reductase 1c1 (Akr1c1) as the first mutated gene in a family with nonsyndromic primary lipedema. Int J Mol Sci 21, 1-13 (2020).
5. Katzer, K., Hill, J. L., McIver, K. B. & Foster, M. T. Lipedema and the potential role of estrogen in excessive adipose tissue accumulation. International Journal of Molecular Sciences vol. 22 Preprint at https://doi.org/10.3390/ijms222111720 (2021).
6. Katzer, K., Hill, J. L., McIver, K. B. & Foster, M. T. Lipedema and the potential role of estrogen in excessive adipose tissue accumulation. International Journal of Molecular Sciences vol. 22 Preprint at https://doi.org/10.3390/ijms222111720 (2021).
7. Katzer, K., Hill, J. L., McIver, K. B. & Foster, M. T. Lipedema and the potential role of estrogen in excessive adipose tissue accumulation. International Journal of Molecular Sciences vol. 22 Preprint at https://doi.org/10.3390/ijms222111720 (2021).
8. McCallion, A. et al. Estrogen mediates inflammatory role of mast cells in endometriosis pathophysiology. Front Immunol 13, (2022).
9. Branco, A. C. C. C., Yoshikawa, F. S. Y., Pietrobon, A. J. & Sato, M. N. Role of Histamine in Modulating the Immune Response and Inflammation. Mediators of Inflammation vol. 2018 Preprint at https://doi.org/10.1155/2018/9524075 (2018).
10. Zierau, O., Zenclussen, A. C. & Jensen, F. Role of female sex hormones, estradiol and progesterone, in mast cell behavior. Frontiers in Immunology vol. 3 Preprint at https://doi.org/10.3389/fimmu.2012.00169 (2012).
11. Goldstein, N. et al. Higher Mast Cell Accumulation in Human Adipose Tissues Defines Clinically Favorable Obesity Sub-Phenotypes. Cells 9, (2020).
12. Elieh Ali Komi, D., Shafaghat, F. & Christian, M. Crosstalk Between Mast Cells and Adipocytes in Physiologic and Pathologic Conditions. Clinical Reviews in Allergy and Immunology vol. 58 388-400 Preprint at https://doi.org/10.1007/s12016-020-08785-7 (2020).
13. Chatterjea, D. & Martinov, T. Mast cells: Versatile gatekeepers of pain. Molecular Immunology vol. 63 38-44 Preprint at https://doi.org/10.1016/j.molimm.2014.03.001 (2015).
14. Shi, M. A. & Shi, G. P. Different roles of mast cells in obesity and diabetes: Lessons from experimental animals and humans. Frontiers in Immunology vol. 3 Preprint at https://doi.org/10.3389/fimmu.2012.00007 (2012).
15. Aksoy, H., Karadag, A. S. & Wollina, U. Cause and management of lipedema-associated pain. Dermatologic Therapy vol. 34 Preprint at https://doi.org/10.1111/dth.14364 (2021).
16. Wang, J. & Shi, G. P. Mast cell stabilization: Novel medication for obesity and diabetes. Diabetes/Metabolism Research and Reviews vol. 27 919-924 Preprint at https://doi.org/10.1002/dmrr.1272 (2011).
17. Kaur, T. & Buttar, H. Mast Cell Stabilizers Cromoglycate And Ketotifen Mitigate High Fat Diet-Induced in Rats. Cardiac Remodeling https://www.researchgate.net/publication/297698767 (2016).
18. Bais, S., Kumari, R., Prashar, Y. & Gill, N. S. Review of various molecular targets on mast cells and its relation to obesity: A future perspective. Diabetes and Metabolic Syndrome: Clinical Research and Reviews vol. 11 S1001-S1007 Preprint at https://doi.org/10.1016/j.dsx.2017.07.029 (2017).
19. Yurdakul Ertan, H. et al. The Investigation of Therapeutic Implications of Mast Cell Stabilizer Cromolyn Sodium on Cholestasis and Cholestatic Pruritus in Experimental Cholestasis. The Turkish Journal of Gastroenterology (2022) doi: 10.5152/tjg.2022.21744.
20. Gao, Y. et al. Aloe-emodin, a naturally occurring anthraquinone, is a highly potent mast cell stabilizer through activating mitochondrial calcium uniporter. Biochem Pharmacol 186, (2021).
21. Li, X. et al. Network pharmacology-based analysis of the mechanism of Saposhnikovia divaricata for the treatment of type I allergy. Pharm Biol 60, 1224-1236 (2022).
22. Sinniah, A. et al. Endogenous annexin-A1 negatively regulates mast cell-mediated allergic reactions. Front Pharmacol 10, (2019).

### BRIEF DESCRIPTION OF THE FIGURES

The novel features believed to be characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 shows staining with Hematoxylin Eosin.

FIG. 2 shows immunohistochemistry with mAb CD117

FIG. 3 shows immunohistochemistry with mAb anti-tryptase

FIG. 4 shows immunohistochemistry with mAb anti-tryptase

### DETAILED DESCRIPTION AND PREFERRED EMBODIMENTS OF THE INVENTION

As used herein, the expression “at least” means one or more and thus includes individual ingredients or components as well as mixtures/combinations of ingredients or components. Other than in the operating examples, or where otherwise indicated, all numbers expressing quantities of ingredients and/or reaction conditions are to be understood as being modified in all instances by the term “about,” meaning within range of the indicated quantity including the number itself.

In some embodiments, it is described a pharmaceutical inhibitor comprised of a mast cell stabilizer for the treatment of lipedema in a subject.

Mast cell stabilizers are a class of medications that are designed to inhibit the release of substances from mast cells, which are a type of white blood cell found in various tissues throughout the body. These cells play a role in immune system responses and contain a variety of substances, including histamine, which can be released when the cells are activated. Mast cell stabilizers are used to treat a range of conditions in which mast cells are involved, including allergies, asthma, and certain types of cancer. These medications are often used prophylactically to reduce the frequency and severity of allergic reactions and asthma attacks and may be used alone or in combination with other medications such as antihistamines or inhaled corticosteroids. It is known that mast cell stabilizers may be able to reduce inflammation and improve insulin sensitivity in individuals with obesity. This is because they work by inhibiting the release of chemicals such as histamine from mast cells, which can have an effect on adipocytes and other tissues in the body.

Sodium cromoglycate, also known as cromolyn sodium, is a mast cell stabilizer that is used to prevent the release of substances from mast cells. It is used to prevent and treat allergic reactions, including allergic rhinitis (hay fever), asthma, and allergic conjunctivitis (eye allergy). Sodium cromoglycate is often used prophylactically to reduce the frequency and severity of allergic reactions and asthma attacks and may be used alone or in combination with other medications. Sodium cromoglycate is generally considered to be safe when used as directed, although it may cause side effects such as stomach upset, nausea, vomiting, and diarrhea in some individuals. It may also cause allergic reactions, including hives, rash, itching, and difficulty breathing.

Further, and in addition there are several other substances known to be mast cell inhibitors, in addition to sodium cromoglycate. Some examples include:
1. Nedocromil: Often used to treat asthma and allergic conjunctivitis.
2. Ketotifen: An antihistamine also used to prevent the release of chemicals from mast cells, making it effective in the treatment of allergies and asthma.
3. Disodium chromoglycate: A salt form of sodium cromoglycate that is also used as a mast cell inhibitor.
4. Olopatadine: An antihistamine that is used to treat allergy symptoms such as itching, tearing, and redness of the eyes. It is also a mast cell inhibitor.
5. Amlexanox: An anti-inflammatory agent used to treat canker sores that has also been shown to be effective as a mast cell inhibitor.

### Scientific Rationale

The scientific premise behind the invention is that targeting mast cells with mast cell stabilizers could be a potential treatment for lipedema. The key points that support the mast cell hypothesis (and histamine) as a key factor in lipedema are as follows:

Firstly, estrogen has an impact on immune system function travels through the body and activates certain cells like mast cells. Mast cells are a type of white blood cell that are found in various tissues throughout the body and play a role in immune system responses. They contain and secrete various substances, including histamine, which can be released when the mast cells are activated. The receptors for estrogen on mast cells are called estrogen receptors.

Secondly, high estrogen concentrations can lead to the activation of mast cells and the release of substances from these cells, a process known as degranulation. One of the substances that is released during mast cell degranulation is histamine, a chemical that plays a role in immune system responses and can cause allergic reactions and inflammation. When mast cells are activated and histamine is released, it can bind to receptors on various tissues and cause a range of symptoms, including swelling, itching, redness, and difficulty breathing. High concentrations of histamine can lead to an exaggerated immune response and can be harmful to the body.

In some aspects, histamine may have an effect on estrogen levels in the body. It is suggested that histamine can stimulate the production and release of estrogen from the ovaries and other estrogen-producing tissues, such as the adrenal glands and adipose tissue. This increase in estrogen production and release can lead to an increase in estrogen concentrations in the body. In addition to its effects on estrogen production, histamine may also affect estrogen metabolism and clearance. Estrogen is metabolized and eliminated from the body through a process called conjugation, in which it is combined with a substance called glucuronide. Some studies have suggested that histamine may inhibit the conjugation of estrogen, leading to increased estrogen concentrations in the body.

Further, high histamine levels in adipose tissue have been found to correlate with proliferation of adipose tissue. This means that an increase in histamine levels within adipose tissue is associated with an increase in the size or mass of the adipose tissue. This may be because histamine may stimulate the growth and proliferation of cells within the adipose tissue, leading to an increase in its size.

Specifically, Lipedema is characterized by the symmetrical accumulation of excess fat in the SAT, especially in the lower limbs. It is distinct from obesity, as it can affect individuals of normal weight and is much more common in women. Unlike obesity, lipedema does not respond to diet and exercise interventions, suggesting that it has a different underlying etiology.

Research suggests that hormonal imbalances, particularly in estrogen effects, as a potential cause of lipedema. These hormonal changes may lead to the abnormal accumulation of SAT and the development of other characteristic symptoms of the disease. Lipedema falls under the classification of localized disorders of SAT, along with conditions such as lipodystrophies, lipomas, and Madelung's disease. Symptoms of lipedema may include pain in the affected areas, altered histology of the fat tissue with the presence of dilated and leaky blood capillaries, collagen deposition, and necrosis. It is also often associated with changes in the menstrual cycle, premenstrual syndrome, painful menstruation, and anxiety.

Some studies suggest that there is involvement of mast cells and histamine in the development and progression of lipedema has been hypothesized. Histological analyses of the diseased adipose tissue revealed an abnormal presence of mast cells, which may be involved in the pathology of the condition. Additionally, high levels of histamine and its metabolites have been found in the tissue of lipedema patients, suggesting that mast cell degranulation and the release of histamine may play a role in the disease. As such, molecules with mast cell-stabilizing effects, such as disodium cromoglycate, may be useful as therapeutic targets for the treatment of lipedema.

### Experiment Study 1

This experiment study involved patients who were diagnosed with lipedema. The study began with collection of a sample from each subject in the study, and three patients received a treatment regimen consisting of twice daily oral doses of sodium cromoglycate (500 mg) for 15 days, as well as a topical cream containing 4% sodium cromoglycate. The effects of this treatment on the patients' symptoms and mast cell activation were evaluated as part of the study.

Thereafter, metabolomic analysis was performed by mass spectrometry. Specifically, the ion-mobility mass spectrometry (IM-MS) technique combined ion mobility with mass spectrometry, allowing different compounds to be characterized together with a specific m/z ratio. SACI-CIMS was used as ionization technique. Data were acquired through the SANIST platform. Values under the Limit of Quantification (LoQ=1 pg/mg) were considered as 0 pg/mg.

Thereafter, histological analysis was performed, wherein the staining with Hematoxylin Eosin allows mast cells to be correctly highlighted, at high magnification and on very thin sections, due to the granule-rich cytoplasm. Very specific and sensitive staining of mast cells is also possible in immunohistochemistry with the CD117 antibody that highlights the proto-oncogene KIT, a growth factor for mast cells. Finally, a specific staining to determine mast cell activation uses mAb anti-tryptase, which targets a protein that is released during degranulation.

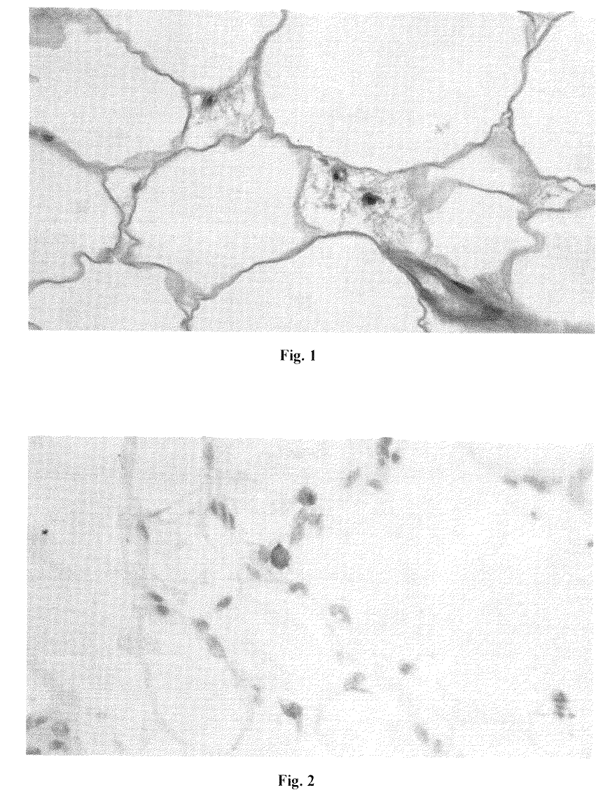

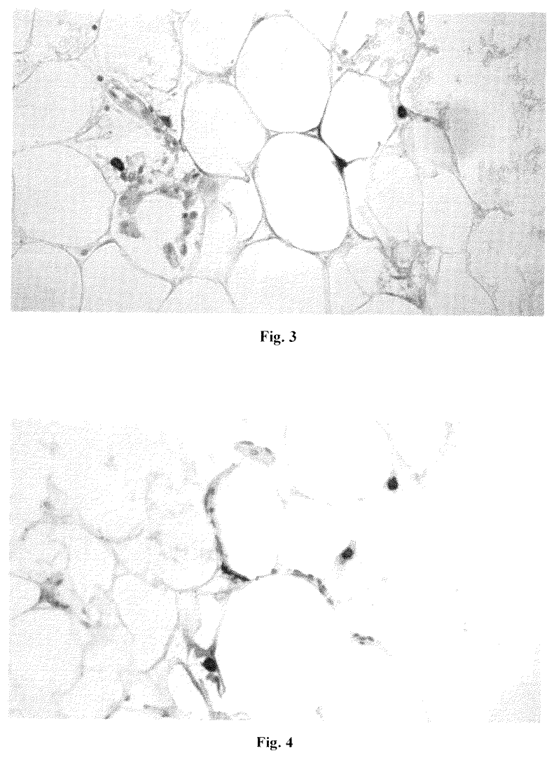

Thereafter, a target histological and metabolomic examination was performed. Specifically, biopsies of subcutaneous adipose tissue were taken from a total of six patients with lipedema and four healthy individuals. The tissue samples from the lipedema patients showed infiltration of mast cells, and the specific staining with mAb anti-tryptase identified activated mast cells, suggesting a potential role of these cells in the development and progression of the disease as shown in FIG. 1-4. Specifically, FIG. 1 shows staining with Hematoxylin Eosin, while FIG. 2 shows immunohistochemistry with mAb CD117, while FIG. 3 shows immunohistochemistry with mAb anti-tryptase and FIG. 4 shows immunohistochemistry with mAb anti-tryptase.

Metabolomic analysis of the tissue samples revealed elevated levels of histamine and its metabolites in the adipose tissue of samples from the lipedema patients as shown in Table 1 below. However, when three lipedema patients were treated with disodium cromoglycate for two weeks, their tissue samples showed normal levels of histamine. This suggests that the mast cell stabilizing properties of disodium cromoglycate may be effective in reducing the levels of histamine and potentially improving the symptoms of lipedema. Finally, tissue samples from the healthy control subjects showed normal levels of histamine, indicating that the higher histamine levels may be abnormal in the adipose tissue of people living with lipedema. These findings support the hypothesis that mast cells and histamine may be involved in the pathology of lipedema and suggest that targeting these pathways may be a promising approach for the treatment of the disease.

TABLE 1
Tissue levels of histamine and its metabolites in the
tissue of people with lipedema, healthy controls and
people with lipedema treated with sodium cromoglycate.

| | | | Lipedema |
|---|---|---|---|
| | | | tissue of |
| | | | patients |
| | | | treated with |
| | Lipedema | Healthy | sodium |
| | tissue | tissue | cromoglycate |
| | (n = 3) | (n = 4) | (n = 3) |
| Histamine | 18.00 ± 16.70 | 0 | 0 |
| (pg/mg) | | | |
| Histidine | 449.00 ± 389.66 | 29.75 ± 21.82 | 20.67 ± 17.00 |
| (pg/mg) | | | |
| 1-Methylhystamine | 3.33 ± 5.77 | 1.50 ± 3.00 | 13.67 ± 10.02 |
| (pg/mg) | | | |
| Imidazole | 5.50 ± 7.78 | 1.25 ± 2.50 | 0 |
| Acetaldehyde | | | |
| (pg/mg) | | | |

### Experiment Study 2

This experiment study involved recruitment and classification of patients who were diagnosed with lipedema into four stages based on the severity of their symptom.

Thereafter, a Metabolomic analysis was performed by mass spectrometry. Specifically, the ion-mobility mass spectrometry (IM-MS) technique combined ion mobility with mass spectrometry, allowing different compounds to be characterized together with a specific m/z ratio. SACI-CIMS was used as ionization technique. Data were acquired through the SANIST platform. Values under the Limit of Quantification (LoQ=1 pg/mg) were considered as 0 pg/mg.

Further, biopsies of subcutaneous adipose tissue were taken from lipedema patients. A total of 44 pathologic adipose tissue 18 healthy adipose tissue were analyzed for this study. Analysis of the pathological adipose tissue samples revealed elevated levels of histamine and its metabolites compared to the healthy adipose tissue samples as seen in Table 2 below.

TABLE 2
Levels of histamine and its metabolites in adipose
tissues with lipedema and healthy adipose tissue.

| | Lipedema | Healthy | |
|---|---|---|---|
| | tissue | tissue | |
| | (n = 44) | (n = 18) | P-value |
| Histamine | 3.00 ± 0.61 | 1.38 ± 0.26 | 1.98E−21 |
| (pg/mg) | | | |
| Histidine | 362.32 ± 54.49 | 173.99 ± 33.84 | 8.34E−22 |
| (pg/mg) | | | |
| 1-Methylhystamine | 1.92 ± 0.36 | 0.95 ± 0.15 | 3.47E−22 |
| (pg/mg) | | | |
| Imidazole | 5.11 ± 0.81 | 2.47 ± 0.45 | 2.15E−22 |
| Acetaldehyde | | | |
| (pg/mg) | | | |

The experiments studies 1 and 2 provide supporting evidence that mast cells and histamine may play a role in the development and progression of lipedema, a chronic and debilitating disease that currently has no cure. The findings from biopsies of SAT from patients with lipedema showed infiltration of mast cells and elevated levels of histamine, which suggests that these factors may be involved in the pathology of the disease. The ability to reduce histamine levels in the tissue of subjects with lipedema after treatment with the mast cell stabilizing agent, disodium cromoglycate, further support this hypothesis and suggest that targeting these pathways may be a promising approach for the treatment of lipedema.

In conclusion, the experiment studies identified the presence of activated mast cells in the adipose tissue of lipedema patients and have shown that these cells may be involved in the pathological processes underlying the disease. The study further demonstrated elevation of tissue histamine levels in the adipose tissue of lipedema patients. In addition, treatment with sodium cromoglycate, a mast cell stabilizing agent, can effectively reduce histamine levels in lipedema patients. This is the first report of the use of sodium cromoglycate as a therapeutic option for lipedema.

### INDUSTRIAL APPLICATION

The industrial application of the invention is the development and production of a pharmaceutical composition or treatment regimen for treating lipedema in a subject using a mast cell stabilizer. The invention has potential to be used in the medical and pharmaceutical industries, providing a new and effective treatment option for individuals suffering from lipedema.

What is claimed is:

1. A pharmaceutical inhibitor for treating lipedema in a subject comprising a therapeutically effective amount of a mast cell stabilizer.

2. The pharmaceutical inhibitor of claim 1, wherein the mast cell stabilizer is selected from sodium cromoglycate, nedocromil, ketotifen, disodium chromoglycate, olopatadine, and amlexanox.

3. The pharmaceutical inhibitor of claim 2, wherein the sodium cromoglycate comprises a composition of about 500 mg.

4. The pharmaceutical inhibitor of claim 2, wherein the sodium cromoglycate comprises a topical cream with a 4% concentration.

5. The pharmaceutical inhibitor of claim 3, wherein the sodium cromoglycate composition is administered orally.

6. The pharmaceutical inhibitor of claim 4, wherein the sodium cromoglycate cream is administered of epicutaneous route.

7. The pharmaceutical inhibitor of claim 5, wherein the treatment regimen further comprises of an epicutaneous administered topical cream containing 4% sodium cromoglycate.

8. The pharmaceutical inhibitor of claim 2, wherein the treatment participates in the reduction of histamine levels in a subject with lipedema.

9. The pharmaceutical inhibitor of claim 2, wherein the treatment participates in the reduction of mast cell degranulation.

10. The pharmaceutical inhibitor of claim 8, wherein the reduction of histamine levels inhibits increase in the size or mass of adipose tissue.

11. The pharmaceutical inhibitor of claim 8, wherein the reduction of histamine levels inhibits the growth and proliferation of cells within the adipose tissue, preventing an increase in its size.

12. A method of manufacture of a pharmaceutical composition comprising steps of:
a. identifying an inhibitor to an active ingredient to the subject;
b. adding an effective amount of a mast cell stabilizer selected from sodium cromoglycate, nedocromil, ketotifen, disodium chromoglycate, olopatadine, and amlexanox; and
c. mixing the preparation for treating lipedema in a subject.

13. A method of using mast cell stabilizer as an inhibitor for treating the proliferation of lipedema, wherein the mast cell stabilizer is selected from sodium cromoglycate, nedocromil, ketotifen, disodium chromoglycate, olopatadine, and amlexanox.

14. The method according to claim 13, wherein the inhibitor participates in the reduction of histamine levels in a subject with lipedema.

15. The method according to claim 13, wherein the inhibitor participates in the reduction of mast cell degranulation.

16. The method according to claim 14, wherein the reduction of histamine levels inhibits increase in the size or mass of adipose tissue.

17. The method according to claim 14, wherein the reduction of histamine levels inhibits the growth and proliferation of cells within the adipose tissue, preventing an increase in its size.

18. The method of claim 13, wherein the sodium cromoglycate comprises a composition of about 500 mg.

19. The method of claim 13, wherein the sodium cromoglycate comprises a topical cream with a 4% concentration.

20. The method of claim 13, wherein the treatment regimen further comprises of an orally administered 500 mg of sodium cromoglycate and an epicutaneous administered topical cream containing 4% sodium cromoglycate.
